(19) United States
(12) Patent Application Publication
(10) Pub. No.: US 2024/0189331 A1
(43) Pub. Date: Jun. 13, 2024

(54) NOVEL TRYPTAMINE ORAL FILM FORMULATION

(71) Applicant: INTELGENX CORP., St-Laurent (CA)

(72) Inventors: Mobarik BILAL, Blainville (CA); Billal TIR, Montreal (CA); Nadine PAIEMENT, St-Laurent (CA)

(73) Assignee: INTELGENX CORP., St-Laurent, QC (CA)

(21) Appl. No.: 18/276,918

(22) PCT Filed: Feb. 14, 2022
(86) PCT No.: PCT/CA2022/050212

§ 371 Date: Aug. 11, 2023

Related U.S. Application Data
(60) Provisional application No. 63/149,215, filed on Feb. 12, 2021.
(60) Provisional application No. 63/171,524, filed on Apr. 6, 2021.
(60) Provisional application No. 63/190,131, filed on May 18, 2021.
(60) Provisional application No. 63/195,680, filed on Jun. 1, 2021.

(51) Int. Cl. A61K 31/675; A61K 9/00; A61K 31/4045; A61K 47/02; A61K 47/10; A61K 47/12; A61K 47/18; A61K 47/22; A61K 47/26; A61K 47/32; A61K 47/38

(52) U.S. Cl. CPC A61K 31/675; A61K 9/006; A61K 31/4045; A61K 47/02; A61K 47/10; A61K 47/12; A61K 47/186; A61K 47/22; A61K 47/26; A61K 47/32; A61K 47/38

(57) ABSTRACT

The present disclosure relates to oral film formulations, which provide a tryptamine active agent. The disclosure more particularly relates to oral film formulations with improved buccal absorption of tryptamine active agents. The present disclosure also relates to formulations and methods for treatment of various symptoms in a patient, such as the treatment of mental health conditions.

### CROSS-REFERENCE TO RELATED APPLICATIONS

The present application claims priority to U.S. Provisional Patent Application No. U.S. 63/149,215 which was filed on Feb. 12, 2021, U.S. Provisional Patent Application No. U.S. 63/171,524, which was filed on Apr. 6, 2021, US Provisional Patent Application No. U.S. 63/190,131, which was filed on May 18, 2021, and U.S. Provisional Patent Application No. U.S. 63/195,680, which was filed on Jun. 1, 2021, the contents of which are incorporated herein by reference in their entirety.

### FIELD OF THE DISCLOSURE

This disclosure relates to oral film dosage formulations and processes for preparing oral film dosage forms, and more particularly to the preparation of oral film dosage forms containing tryptamines, and preferably psilocybin, psilocin, norpsilocin, bufotenin, bufotenidine, baeocystin, norbaeocystin, and aeruginascin, and salts thereof, and combinations thereof. This disclosure further relates to a method of treating various diseases, afflictions and disorders with the administration of a tryptamine oral film formulation bucally.

### BACKGROUND OF THE DISCLOSURE

It is often desirable to administer a pharmaceutical ingredient using an oral film dosage form. Oral film dosage forms have several advantages when compared with tablet and capsules. Many people have difficulty swallowing tablets and capsules, and risk choking while attempting to swallow solid oral dosage forms, but can self-administer a film dosage form without difficulty.

Oral films refer to a type of dosage form that is distinctly different from pills, tablets, caplets, and capsules, and in which the dosage form is a thin strip of material. It will be understood that the term “oral film” and “oral film formulation” includes delivery systems of any thickness, including films, film strips, discs, sheets, and the like, in any shape. Such films are typically rapidly disintegrating or rapidly dissolving, but can also exhibit longer disintegration times when required. The films are generally sufficiently flexible to allow bending or even folding without breaking. For example, the films typically have length and width dimensions on the order of 5 to 35 mm, although larger or smaller dimensions are possible and may be desirable in particular circumstances, and a thickness on the order of 5 to 300 μm, although larger or smaller thicknesses are possible and may be desirable in certain circumstances.

Tryptamines and more specifically psilocybin and its derivatives are an important group of compounds with various medicinal possibilities. Tryptamine is an indolamine metabolite of the essential amino acid, tryptophan. The chemical structure is defined by an indole—a fused benzene and pyrrole ring, and a 2-aminoethyl group at the third carbon. The structure of tryptamine is a shared feature of certain aminergic neuromodulators including melatonin, serotonin, bufotenin and psychedelic derivatives such as dimethyltryptamine (DMT), psilocybin, psilocin and others.

Tryptamines can be found in nature in plants, fungi, animals, microbes and amphibia. Its importance as the neurotransmitter, neuromodulator and as psychedelic drugs is well known The psychedelic and medicinal effects of some plants and fungi have been known and deliberately exploited by humans for thousands of years. Tryptamines are indolamine metabolites of the essential amino acid, tryptophan. The chemical structure is defined by an indole—a fused benzene and pyrrole ring, and a 2-aminoethyl group at the third carbon. The structure of tryptamine is a shared feature of certain aminergic neuromodulators including melatonin, serotonin, bufotenin and psychedelic derivatives.

The effects caused by the administration of tryptamines are closely related to their structures, as each of these compounds has a different receptor affinity to which are related psychoactive phenomena. Tryptamines show agonist action at multiple receptors including 5HT2a-1a-2c, serotonin receptors, and several ion channels. (Ray T. S. Psychedelics and the Human Receptorome. 2010.)

The metabolism of the amino acid L-tryptophan is a highly regulated physiological process leading to the generation of several neuroactive compounds within the central nervous system. These include the aminergic neurotransmitter serotonin (5-hydroxytryptamine, 5-HT), products of the kynurenine pathway of tryptophan metabolism (including 3-hydroxykynurenine, 3-hydroxyanthranilic acid, quinolinic acid and kynurenic acid), the neurohormone melatonin, several neuroactive kynuramine metabolites of melatonin, and the trace amine tryptamine. The integral role of central serotonergic systems in the modulation of physiology and behaviour has been well documented since the first description of serotonergic neurons in the brain some 40 years ago.

Several classifications have been suggested for these substances. Tryptamines can be divided into two main groups: the simple tryptamines, including dimethyltryptamine (DMT), and the ergolines (a group of chemical compounds that were originally synthesized from a fungus ergot, among which is the lysergic acid diethylamide (LSD). Tryptamines have been further subdivided into three subgroups: (1) simple tryptamines, without modification of the indole ring; (2) tryptamines having a modification on the 4-position on the indole ring; (3) tryptamines having a modification on the 5-position. Only substitutions on the 4-5 positions were considered in such a subdivision because changes in position 6 or 7 resulted in reduced hallucinogenic activity. (Hill S. L, Thomas S. H. Clinical Toxicology of newer recreational drugs. Clin. Toxicol. 2011; 49(8):705-719. doi: 10.3109/15563650.2011.615318.)

When the drug is administered orally, only about 50% of the drug is absorbed into the bloodstream because of the first pass metabolism effect and the maximum plasma concentration occurring only after approximately 80 minutes. To improve such pharmacokinetic data, there is a need for the development of a stable psilocybin formulation where the absorption of the drug takes place in the mouth through the buccal mucosa, formulations and processes for doing so will be disclosed herein. Such a formulation will enable the drug to reach the systemic circulation directly limiting the hepatic metabolism. The result will include several therapeutic benefits such as faster onset of action, increased bioavailability, and lessened drug side effects such as nausea and panic attacks.

US patent 20210316347A1 describes pharmaceutical formulations of novel indole compounds and psilocybin analogs. However, the invention discloses only such examples of pharmaceutical compositions using dermal patches.

US patent 20210015738A1 describes an oral dissolvable film formulation that includes a psychedelic compound such as psilocybin, psilocin and baeocystin. The ability of the film to deliver the drug through the buccal transmuccosal membrane was not disclosed. The disclosure mentions only broadly that the active ingredient of only up the 5 mg can enter the blood stream enterically, mucosally, buccally, gingivally, and/or sublingually as the film dissolves in the mouth, but provides no practical or descriptive methods for doing so.

Drug delivery via oral mucosa is an alternative method of systemic administration for various classes of therapeutic agents. Among the oral mucosae, buccal and sublingual mucosae are the primary focus for drug delivery. Buccal delivery offers a clear advantage over the peroral route by avoidance of intestinal and hepatic first-pass metabolism. However, despite offering the possibility of improved systemic drug delivery, buccal administration has been utilized for relatively few pharmaceutical products so far. One of the major limitations associated with buccal delivery is low permeation of therapeutic agents across the mucosa. It is appreciated that those of skill in the art understand that not all oral film strips have the potential to deliver a given drug directly into the blood stream via the sublingual, buccal, gingival or palatal route. Besides the biochemical characteristics of the buccal membranes, which are responsible for the barrier function and permeability, various factors of the given drug molecule influence the extent of permeation through the membranes: the lipid solubility, the water solubility of the drug, its degree of ionization, pKa of the drug, pH of the drug solution, presence of saliva, molecular weight and size of the drug, various physicochemical properties of the formulation, and the presence or absence of permeation enhancers.

US2021036310 describes new chemical analogues of tryptamine molecules by substituting some hydrogen atoms and alkyl molecules by deuterium and methods for treating patients with specific disease. Buccal absorption of psilocybin is not addressed.

These and other inefficiencies and opportunities for improvement are addressed and/or at least partially overcome by the systems, assemblies and methods of the present disclosure.

### SUMMARY OF THE DISCLOSURE

In one aspect of the disclosure, a tryptamine or tryptamine derivative is the active agent in an oral film formulation further comprising an acidifying agent, a penetration enhancer and a plasticizer, all of which enhance the permeation of the active agent when administered bucally.

In certain embodiments, the psilocybin analogues and combinations thereof provided herein include any compound that is structurally related to psilocybin and functionally mimics and/or antagonizes the action of serotonin.

In certain embodiments of the inventive oral film formulation, psilocybin and psilocybin analogues and combinations thereof may be derived synthetically or bioengineered; or extracted from naturally occurring mushrooms that have been well described in the art.

In one aspect of the disclosure, the disclosure pertains to the oral film formulation comprising an active agent selected from psilocybin, psilocin, norpsilocin, bufotenin, bufotenidine, baeocystin, norbaeocystin, and aeruginascin, and salts thereof, and combinations thereof, a penetration enhancer, an acidifying agent and a plasticizer, wherein the pH range of the oral film formulation is preferably between 1.5 and 5.

In certain aspects of the disclosure, the oral film formulation has a surface pH range is between 2 and 4.5, preferably between 2.5 and 4 or more preferably between 3 and 3.5.

In one aspect of the disclosure, the oral film formulation employs a penetration enhancer is a quaternary ammonium salt selected from benzalkonium chloride, cetylpyridinium chloride, cetrimide, and combinations thereof.

In one aspect of the disclosure, the oral film formulation employs an acidifying agent is selected from phosphoric acid, citric acid, tartaric acid, malic acid, acetic acid, succinic acid, maleic acid, hydrochloric acid, benzoic acid, fumaric acid, glucoronic acid, or lactic acid, and is preferably citric acid or phosphoric acid.

In one aspect of the disclosure, the oral film formulation of claim 1, the penetration enhancer is present in an amount of from about 0.1% to about 10% by weight, 0.5% to 5%, or 1% to 3% by weight relative to the total dry weight of the oral film formulation, preferably 0.5% to 5%.

In one aspect of the disclosure, the active agent is present in an amount of from about 2 to 25% by weight or about 5 to 20% by weight, relative to the total dry weight of the oral film formulation.

In one aspect of the disclosure, the oral film formulation further comprises a mucoadhesive agent, wherein the mucoadhesive agent is selected from the group of hydroxypropylmethyl cellulose, hydroxyethyl cellulose, hydroxypropyl cellulose, sodium alginate, sodium carboxymethyl cellulose, guar gum, karya gum, methylcellulose, polyethylene oxide, retene, tragacanth, and combinations of two or more thereof.

In one aspect of the disclosure, a mucoadhesive agent is present in an amount of from about 0.5% to about 20% by weight, or about 1% to about 5% by weight, relative to the total dry weight of the oral film formulation.

In one aspect of the disclosure, the plasticizer is present in an amount up to 25% by weight, such as from 0.5% to 25%, 1% to 20%, 2% to 15% or 5% to 10% by weight, relative to the total dry weight of the oral film formulation.

In one aspect of the disclosure, the oral film formulation further comprises an antioxidant, wherein the antioxidant is present in an amount of from about 0.01% to 5% or 0.1% to 1% by weight, relative to the total dry weight of the oral film formulation.

In one aspect of the disclosure, the oral film formulation further comprises at least one film forming polymer, wherein the at least one film forming polymer is selected from the group of hypromelose HPMC E50, copovidone (copolymers of N-vinyl-2-pyrrolidone and vinyl acetate), povidone (polyvinylpyrrolidone), polyethylene oxide, polyvinyl alcohol, polyethylene glycol, polyacrylic acid, methylmethacrylate copolymer, carboxyvinyl copolymers, polydextrose, pullulan, hydroxypropylmethyl cellulose, hydroxyethyl cellulose, hydroxypropyl cellulose, carboxymethyl cellulose, sodium alginate, xanthan gum, tragancath gum, guar gum, acacia gum, arabic gum, starch, gelatin, and combinations of two or more thereof.

In one aspect of the disclosure, the film forming polymer or combination of film forming polymers is present in an amount of from 10% to 90% by weight, 20% to 80% by weight, or 30% to 70% by weight, relative to the total dry weight of the oral film formulations.

In one aspect of the disclosure, the oral film formulation further comprises a flavor.

In one aspect of the disclosure, the oral formulation further comprises a flavor enhancer.

In one aspect of the disclosure, the oral formulation further comprises a pore former.

In one aspect of the disclosure, the oral formulation further comprises a preservative agent.

In one aspect of the disclosure, the oral formulation further comprises a sweetener.

In one aspect of the disclosure, a method is disclosed for treating depression, anxiety, migraines, addiction, dementias, Alzheimer's disease, eating disorders, obsessive compulsive disorder, Lyme disease syndrome, post-traumatic stress disorder, abuse disorders including opioid addiction, alcohol addiction, nicotine addiction, cannabinoid addiction, headache, central nervous system inflammation, and disorders of cognition and memory with the disclosed oral film formulation containing psilocybin and derivatives thereof, wherein the method comprises administrating the oral film formulation buccally.

In one aspect of the disclosure, a method is disclosed of using an acidifying agent and penetration agent for enhancing permeation of psilocybin, psilocin, norpsilocin, bufotenin, bufotenidine, baeocystin, norbaeocystin, and aeruginascin, and salts thereof, and combinations thereof, wherein the method comprises administering the oral film formulation buccally.

In one aspect of the disclosure, it is disclosed an oral film formulation comprising an active agent, a penetration enhancer, an acidifying agent and a plasticizer, wherein the surface pH range of the oral film formulation is preferably between 1.5 and 5, and wherein the active agent is a tryptamine.

In one aspect of the disclosure, it is disclosed a method for treating depression, anxiety, migraines, addiction, dementias, Alzheimer's disease, eating disorders, obsessive compulsive disorder, Lyme disease syndrome, post-traumatic stress disorder, abuse disorders including opioid addiction, alcohol addiction, nicotine addiction, cannabinoid addiction, headache, central nervous system inflammation, and disorders of cognition and memory with the oral film formulation containing a tryptamine as an active agent, wherein the method comprises administrating the oral film formulation bucally.

According to one aspect of the disclosure, the oral film formulation comprises a protective backing layer to further increase the local concentration at the membrane interface and minimize ionization of the active agent due to the local pH environment within the oral cavity.

### BRIEF DESCRIPTION OF THE FIGURES

FIG. 1 illustrates psilocybin ionization state and pH

FIG. 2 illustrates a process flow chart for the blend preparation for psilocybin sublingual/buccal film.

FIG. 3 illustrates a process flow chart of coating, drying and packaging of psilocybin sublingual/buccal film.

FIG. 4 illustrates acidic psilocybin films made in phosphoric acid, and demonstrates the effect of permeation enhancers on permeation through porcine buccal mucosa.

FIG. 5 illustrates acidic psilocybin films made in citric acid, and demonstrates the effect of permeation enhancers on permeation through porcine buccal mucosa.

FIG. 6 illustrates non-acidic psilocybin films, and demonstrates the absence of psilocybin permeation through porcine buccal mucosa.

### DETAILED DESCRIPTION

Unless defined otherwise, all technical and scientific terms used herein have the same meaning as commonly understood by one of ordinary skill in the art.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The tryptamines play a fundamental role in human life: serotonin (5-hydroxytryptamine, 5-HT), one of the most important signaling hormones in the body, is a tryptamine natural derivative involved in regulation and modulation of multiple processes within the central nervous system, such as sleep, cognition, memory, temperature regulation and behavior.

The terms “oral dissolving film,” “oral dissolvable film”, “oral disintegrating film”, OSF, “oral soluble film”, “ODF”, “oral chewable film”, “OCF”, “oral thin film”, “OTF,” “oral drug strip” or “oral strip” refer to a product used to administer a predetermined amount of active ingredient(s) via oral administration such as oral transmucosal absorption, sublingual delivery or buccal delivery and will be referred to throughout as oral film(s), and more specifically as an oral film formulation when combined with the various components described herein.

The term “Tryptamine” include N,N-dimethyltryptamine (DMT) and its derivatives alpha-methyltryptamine (AMT), 5-methoxy-N,N-dimethyltryptamine (5-MeO-DMT) and 5-methoxy-N,N-diisopropyltryptamine (5-MeO-DIPT), and psilocin (4-hydroxy-N,N-dimethyltryptamine also known as 4-OH-DMT) and psilocybin ([3-(2-Dimethylaminoethyl)-1H-indol-4-yl]dihydrogen phosphate. In addition to psilocin, other metabolites of psilocybin include; (1) 4-hydroxyindole-3-yl-acetaldehyde (4H1A), (2) 4-hydroxyindole-3-yl-acetic-acid (41-IIAA), and (3) 4-hydroxytryptophol (41-IT).

Common tryptamine structure:

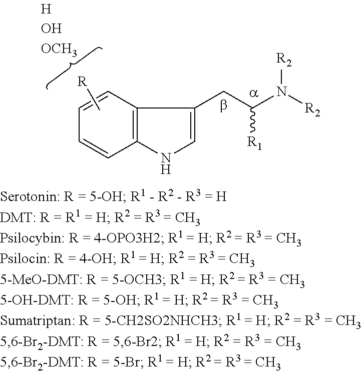

Psilocybin, psilocin, baeocystin, norbaeocystin, and aeruginascin are tryptamines structurally similar to the neurotransmitter serotonin. The therapeutic implications of psilocybin are broad with active clinical studies targeting depression, anxiety, migraines, addiction, dementias, Alzheimer's disease, eating disorders, obsessive compulsive disorder, and palliative care. Pilot studies have reported on the efficacy of psilocybin in, for example, treatment-resistant depression (Carhart-Harris et al., 2016, 2018), major depressive disorder (Davis et al., 2021), terminal-cancer—related anxiety (Griffiths et al., 2016; Grob et al., 2011; Ross et al., 2016), obsessive-compulsive disorder (OCD) (Moreno et al., 2006) and alcohol and nicotine dependence (Bogenschutz et al., 2015; Johnson et al., 2014).

Recent studies on psilocybin, its derivatives and combinations thereof, have been reported to have efficacy in models and small clinical trials of post treatment Lyme disease syndrome, dementias, Alzheimer's disease, post-traumatic stress disorder, anorexia nervosa, depression and anxiety, abuse disorders including opioid addiction, alcohol addiction, nicotine addiction, cannabinoid addiction, headache, central nervous system inflammation, dementia, and disorders of cognition and memory. Psilocybin has also been reported to acutely reduce clinical symptoms of depressive disorders. (Journal of Psychopharmacology, Volume: 36 issue: 1, page(s): 114-125, Jan. 4, 2022)

Psilocybin is a prodrug that turns to its pharmacologically active ingredient psilocin in the body. Psilocybin is an indole alkaloid and a naturally occurring psychoactive that is produced by more than 200 species of mushrooms. It is dephosphorylated in vivo via oral dosing to produce the active compound psilocin. Psilocybin and psilocin are both indole compounds, are known to be potent 5-HT agonists, and can cross the blood-brain barrier. The similarity in structure of psilocin to serotonin is illustrated here:

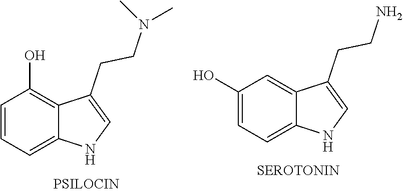

Formulated and administered correctly, psilocin and psilocybin provide fast-acting and long-lasting changes to a person's mood and beyond in term of medicinal effects. These effects can be accomplished with only minor side effects, low potential for addiction, low potential for abuse, and low risk of toxicity. A useful summary of the role of psilocybin in humans can be found in Passie et al. (2002), “The Pharmacology of Psilocybin,” Addiction Biology 7: 357-364. That psilocybin has neurogenerative properties was elucidated by Catlow et al. (2013), “Effects of psilocybin on hippocampal neurogenesis and extinction of trace fear conditioning,” Experimental Brain Research 228: 481-491.

The term “film” refers to a type of dosage form that is distinctly different from pills, tablets, caplets, and capsules, and in which the dosage form is a thin strip of material. It will be understood that the term “film” includes delivery systems of any thickness, including films, film strips, discs, sheets, and the like, in any shape. Such films are typically rapidly disintegrating or rapidly dissolving, but can also exhibit longer disintegration times when required. The films are generally sufficiently flexible to allow bending or even folding without breaking. For example, the films typically have length and width dimensions on the order of 5 to 35 mm, although larger or smaller dimensions are possible and may be desirable in particular circumstances, and a thickness on the order of 5 to 300 μm, although larger or smaller thicknesses are possible and may be desirable in certain circumstances.

The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication. This protective layer also serves to favor the directed absorption of the API within the oral cavity rather than enteric uptake in the gastrointestinal (GI) tract.

The term “mucoadhesive or bioadhesive” means that the composition of the film layer is formulated to adhere to the mucous membrane through which delivery of the active agent is targeted. For example, bioadhesive polymers used in formulating the film should be selected to exhibit adequate adhesion within the environment at the targeted mucous membrane to ensure that the bioadhesive layer remains in contact with the mucous membrane to which it is applied and allows the active agent to directly enter the blood stream through the mucous membrane.

The term “instantly wettable” and variations thereof generally refers to the ability of the film dosage form to rapidly imbibe moisture upon oral administration to a subject and immediately soften, whereby the subject is prevented from experiencing a prolonged adverse feeling in the mouth, and with respect to certain aspects of the disclosure refers to embodiments in which moisture (i.e., water) applied to a surface of the film penetrates the thickness of the film (e.g., typically about 5 μm to 200 μm) within 5, 10, 15 or 20 seconds. The wettability also ensures quick mucoadhesion ensuring the film sticks to the mucosa and stays in place.

The term “cannabinoid” represents a group of C21 terpenophenolic compounds found uniquely in cannabis plants. Cannabinoids include the psychoactive compounds Δ9-tetrahydrocannabinol (THC), Δ8-THC, cannabinol (CBN), 11-hydroxy Δ9-THC, anandamide, and the non-psychoactive compounds cannabidiol (CBD), cannabichromene, and (-) 08-THC-11-oic acid. The term cannabinoid is used herein to refer to a cannabinoid that is either synthetic or extracted from the plant. It is also used to refer to a single cannabinoid or mixture of cannabinoids. The term “cannabis” is used to refer to plants of the genus Cannabis, including Cannabis sativa and Cannabis indica.

The term “mucoadhesive” and variations thereof generally refers to film matrix or pharmaceutical dosage form interacting by means of adhesion with the mucus that covers epithelia.

The “surface pH” is the pH measured on a surface of the film, such as the top or bottom surface of a monolayer film or on an exposed surface of the layer containing the active in a multilayer oral film. The film is prepared for pH testing by slightly wetting the film (adding water as needed for a pH test—e.g. one to three drops). The pH is then measured by bringing the electrode in contact with the surface of the oral film. This measurement of the surface pH is preferably performed on several films of the same formulation.

The term “rapidly disintegrating” and variations thereof generally refers to the ability of the film dosage forms to break up into submicron particles or completely dissolve within an acceptable period of time (e.g., within 60 seconds, within 45 seconds, within 30 seconds, within 20 seconds, or within 15 seconds of being administered, i.e., placed in the oral cavity of a subject).

The terms “blend” or “blending media” and variations thereof generally refers to the combination of the OF formulation with the presence of solvents.

The term “drug absorption” or “absorption” as used in this specification, refers to the process of movement from the site of administration of a drug toward the systemic circulation, e.g., into the bloodstream of a subject.

The term “residence time” as used in the specification refers to the time taken by the film to disintegrate on the buccal mucosa.

Any number of active agents or active pharmaceutical ingredients may be included in the films discussed herein. The term “active(s)” or “active agent(s)” refers mainly to active pharmaceutical ingredients (APIs), but may also refer generally to any agent(s) that chemically interacts with the subject to which it is administered to cause a biological change, such as, but not limited to, eliminating symptoms of disease or regulating biological functions. The term “Pharmaceutical Ingredient or API” and variations thereof generally refers to any agent that is being administered orally to a subject and includes pharmaceutically active agents, nutraceutically active agents, and breath freshening agents. Examples of pharmaceutically active agents include ACE-inhibitors, antianginal drugs, anti-arrhythmics, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, tadalafil, and vardenafil, fertility agents, gastrointestinal agents, homeopathic remedies, hormones, hypercalcemia and hypocalcemia management agents, immunomodulators, immunosuppressives, anti-migraine preparations such as rizatriptan, eletriptan and zolmitriptan, motion sickness treatments, muscle relaxants, obesity management agents, osteoporosis preparations, oxytocics, parasympatholytics, parasympathomimetics, prostaglandins, psychotherapeutic agents, respiratory agents, sedatives such as lorazepam or diazepam, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents such as alprazolam, anti-ulcer agents, anti-inflammatory substances, coronary dilators, cerebral dilators, peripheral vasodilators, psycho-tropics, stimulants, anti-hypertensive drugs, vasoconstrictors, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spadmodics, terine relaxants, anti-obesity drugs, erythropoietic drugs, anti-astmatics, cough suppressants, mucolytics, DNA and genetic modifying drugs, and combinations thereof. Examples of nutraceutically active agents include various dietary supplements, vitamins, minerals, herbs and nutrients.

The term “acceptable dimensions and drug loading” as used herein encompasses a film with dimensions of up to three centimeters by five centimeters (length by with) and two millimeters of thickness and a drug loading ranging from 1.5%-60% of the total weight of the film.

A variety of additives that can be integrated into the films may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added along with the active ingredient(s).

The term “matrix” or “film matrix” refers to the polymer component or mixture of polymers, which creates the film forming matrix supporting the API within the oral film dosage form.

The term “polymer” refers to a long molecule chain made of many repeating units. The choice of polymers in an oral film formulation affect the mechanical and texture properties of the oral film formulation and drug release.

The terms “suspending agent” (also referred to as a “viscosity increasing agent”) refers to water soluble ingredients or non-water soluble ingredients or combination thereof employed to prevent adjacent suspended particles from coming close enough to join each other by increase sufficiently the viscosity of the drug vehicle, and enables by steric stabilization the suspension to be stably maintained, beside above properties certain suspending agent/viscosity increasing agent additionally interact with biological mucosa to create an strengthen oral film mucoadhesion. Examples comprise polysaccharide in the form of one or a mix of Hydroxypropylmethylcellulose (HPMC) where the polymer structure combines both hydrophobic (methoxy group) and hydrophilic substitutions (hydroxypropoxy group) where the 2% aqueous viscosity is between about 1298 to about 5040 millipascal second (mPas) (2%, 20 C), Hydroxypropyl Cellulose (HPC) where the 2% aqueous viscosity is above about 150 mPas (2%, 25 C), hydroxyethyl cellulose (HEC), Gums such as water soluble carboxymethyl cellulose (CMC), Gellan, propylene glycol alginate, water soluble alginate salt, Acacia, Pectin, Xanthan, guar gum, carrageenan, and water insoluble alginates derivatives, water insoluble CMC derivatives, colloidal silicon dioxide, Agar, Locust bean, tragacanth. It may also comprise Polyvinylpyrrolidone of Molecular Weight (MW) of 1 000 000 MW and above (K-value of 85 and above) with aqueous viscosity of 300 mPAs (10%, 20 C) and above and higher molecular weight polyethylene oxide (PEO) (MW above 600 000). The following are excluded from the definition of the terms “suspending agent/viscosity increasing agent”: one or a mix of HPMC where the polymer structure do not combines both hydrophobic and hydrophilic substitutions, and or having aqueous viscosity below 1298 mPas or above 5040 mPas (2%, 20 C), Methyl cellulose (MC), Microcrystalline cellulose (MCC), powdered cellulose, Sodium Starch Glycolate, starch, Polyvinylpyrrolidone of MW below 1.000.000 MW and K-value below 85 and with aqueous viscosity of less than 300 mPAs (10%, 20 C), polyvinylpyrrolidone-vinyl acetate copolymer, polyplasdone crospovidone, HPC where the 2% aqueous viscosity is below 150 mPas (2%, 25 C), water insoluble bentonite.

The term “surfactant” refers to surfactant(s) of an Hydrophilic Lipophilic Balance (HLB) of 7 and higher having an amphiphilic structure, with polar hydrophilic head (ionic or no-ionic) and non-polar hydrophobic tail. Surfactants are employed to dissipate the free surface energy of particles by reducing the interfacial tension and contact angle between the solid and the suspending vehicle and comprise PEG 300 oleic glycerides (Labrafil® M-1944CS), PEG 300 linoleic glycerides (Labrafil® m-2125CS); Hydroxylated lecithin; Caprylocaproyl polyoxyl-8 glycerides; Polyoxyethylene (4) sorbitan monostearate, Polyoxyethylene 20 sorbitan tristearate, Polyoxyethylene (5) sorbitan monooleate, Polyoxyethylene 20 sorbitan trioleate; Sorbitan Esters (Sorbitan Fatty Acid Esters) such as: Sorbitan monolaurate, Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene 20 sorbitan monooleate, Polyoxyethylene 20 sorbitan monoisostearate Polyethylene glycol monostearate (Gelucire 48/16), poloxamer having MW up to 14.600, viscosity up to 3100 mPAs (77 C) but exclude surfactant(s) of an HLB below 7 such as Propylene glycol monocaprylate type I, Propylene glycol monocaprylate type II, Propylene glycol monolaurate, Sorbitan monoisostearate, Sorbitan monooleate, Sorbitan monopalmitate, Sorbitan monostearate, Sorbitan sesquioleate, Sorbitan trioleate, Sorbitan tristearate, glyceryl monoleate.

The term “mucoadhesive film former” refers to polymers that form the film matrix, film strip, film sheet and dissolves in aqueous environment and gives bio-adhesive properties to the mucosa examples comprising PEO, Pullulan, CMC, HPC, HPMC and exclude ethyl cellulose (EC), polyvinyl alcohol (PVA), Starch, Polymethacrylate polymers. Examples of mucoadhesive materials that can be used to prepare the mucoadhesive particles include poly(ethylene oxide), polyvinyl pyrrolidone, poly(acrylic acid) derivatives (e.g., commercially available Carbopol®), polycarbophil, polyoxyalkylene ethers, polymethacrylates, polymethacrylates-based copolymers (e.g., commercially available Eudragit®), biodegradable polymers such as poly(D,L-lactide-co-glycolide) (e.g., commercially available Resomer®), anionic biopolymers such as hyaluronic acid, or sodium carboxymethylcellulose, cationic biopolymers such as chitosan or poly(L-lysine) and other cellulose derivatives. Other mucoadhesive polymers that can be used include methyl vinyl ether-maleic acid, a mixed salt of sodium/calcium methyl vinyl ether-maleic acid, methyl vinyl ether-maleic anhydride, and half esters (monoethyl; monobutyl and isopropyl ester) of methyl vinyl ether-maleic anhydride copolymers (e.g., commercially available Gantrez®).

The term “therapeutically effective amount” refers to an amount of a pharmaceutically active agent, which when administered to a particular subject, considering the subject's age, weight and other relevant characteristics, will attenuate, ameliorate, or eliminate one or more symptoms of a disease or condition that is treatable with the pharmaceutically active agent. Generally, the therapeutically effective amount of bupropion is provided in the commercially available products, and that of mecamylamine is provided in.

The amorphous active agent(s) or solid dispersion containing the amorphous active agent(s) is mixed with the liquid film-forming formulation without dissolving the active agent(s).

The term “suspended” (and variations thereof) refers to a dispersion of solid material (e.g., particles or powder) in a bulk liquid medium, in which the solid material is not completely dissolved on a molecular level, and will eventually settle out of the liquid in the absence of agitation. In a suspension, the suspended material is not dissolved in the liquid.

The term “treating”, “treat” or “treatment” as used herein embraces both preventative, i.e., prophylactic, and palliative treatment, i.e., relieve, alleviate, or slow the progression of the patient's disease, disorder or condition. The term “treat” or “treatment” as used in this specification in the context of a mental health disorder such as addiction, depression, anxiety and posttraumatic stress disorder (PTSD) refers to any treatment of a disorder or disease associated with a mental health disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent”.

The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication or simply against constant washing of the saliva. This protective layer also serves to favor the directed absorption of the API within the oral cavity rather than enteric uptake in the gastrointestinal (GI) tract. The term “mucoadhesive or bioadhesive” means that the composition of the film layer is formulated to adhere to the mucous membrane through which delivery of the active agent is targeted. For example, bioadhesive polymers used in formulating the film should be selected to exhibit adequate adhesion within the environment at the targeted mucous membrane to ensure that the bioadhesive layer remains in contact with the mucous membrane to which it is applied and allows the active agent to directly enter the blood stream through the mucous membrane. Mucoadhesive ingredients are often responsible for the strong bond between the product and the mucosal surface.

The term “penetration enhancer” refers to components that result in buccal penetration enhancement, and result from agents being able to (a) increase the partitioning of drugs into the buccal epithelium, (b) extract (and not disrupt) intercellular lipids, (c) interact with epithelial protein domains, and/or (d) increase the retention of drugs at the buccal mucosal surface. Examples of penetration enhancers are benzalkonium chloride, cetylpyridinium chloride, cetrimide and other quaternary ammonium salts.

The term “acidifying agent” refers to components that can be added to oral dosage forms to lower the pH of a formulation within a desired range. Examples of acidifying agents are citric acid and phosphoric acid.

The term “bulking agent” refers to fillers that are added to film formulations during manufacturing. Bulking agents can improve film texture, uniformity and eventual performance of the final dosage form.

The term “antioxidant” refers to an oxygen scavenger which prevents or reduce oxidative degradation of the API. Examples of oxygen scavengers or antioxidants that substantially improve long-term stability of an oral dosage form against oxidative degradation of the active agent are propyl gallate, EDTA, Copper II chloride, Vitamin E and Sodium metabisulfite.

The expression “antioxidant” as used to describe and claim certain embodiments of the invention refers generally to a chemical compound or substance that inhibits oxidation of a tryptamine or other active agent. Antioxidants include compounds that react with and neutralize free radicals or chemicals that release free radicals and/or which otherwise stop, prevent or retard oxidation reactions that lead to or cause degradation of the cannabinoid in either its free or complexed form. Antioxidants that may be employed in the invention include pharmaceutically acceptable acids, especially pharmaceutically acceptable organic acids such as those selected from C2-C10 alkyl- or alkenyl-carboxylic acids having two or more carboxylic groups. Specific examples include malonic acid, succinic acid, fumaric acid, maleic acid, adipic acid, lactic acid, levulinic acid, sorbic acid, glutamic acid, aspartic acid, oleic acid, glutaric acid, tartaric acid, malic acid, ascorbic acid, and citric acid. Other antioxidants that can be used include butylated hydroxyanisole (BHA) and butylated hydroxytoluene (BHT), as well as other phenolic antioxidants.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or method steps.

The term “disintegrating agent”/pore former refers to a component that helps the matrix of the film to hydrate and disintegrate upon contact with saliva.

The term “refreshing agents”, also called cooling agents, are chemicals that trigger the cold sensitive receptors creating a cold sensation.

The term “flavoring Agent” or flavor” and variations thereof generally refers to concentrated preparations, with or without flavor adjuncts required in their manufacture, used to impart flavor, with the exception of salt, sweet, or acid tastes. Flavoring agents may be classified as natural, artificial, or natural and artificial (N&A) by combining the all natural and synthetic flavors or other forms known in the art. Flavouring agents are categorized by their physical classification as solid flavoring agents and liquid flavoring.

The term “flavor Enhancer” and variations thereof generally refers to compounds that particularly enhance certain tastes or reduce undesirable flavors without having an especially strong taste of their own. They harmonize taste components and make food/drug preparations more palatable. Examples include but are not limited to maltol, ethyl maltol and monosodium glutamate, glutamic acid, glutamates, purine-5-ribonucleotides, inosine, guanosine, adenosine 5_-monophosphates, sugars, sweetener, carboxylic acids (e.g., citric, malic, and tartaric), common salt (NaCl), amino acids, some amino acid derivatives (e.g., monosodium glutamate— MSG), and spices (e.g., peppers) are most often employed, yeast, yeast extract, dried yeast and others or mixtures thereof.

The term “particle” refers to a nanoscopic (1 to 1000 nm) or microscopic (1 to 1000 micrometers) solid or semi-solid aggregate structures. The particles can be spherical or non-spherical (e.g., ellipsoidal or rod-like) structures, with hollow or solid core, such as solid spheres, micelles, vesicles, liposomes or lamellaes. For example, it is possible to employ known techniques to form mucoadhesive particles comprising a mucoadhesive material and an active agent that have a particle size range from a few nanometers (e.g., 5 nm, 10 nm, 50 nm, 100 nm) to a few micrometers (e.g., 1 μm, 100 μm, 200 μm, 300 μm, 500 μm).

The term “sweetener” and variations thereof generally refers to a solid or liquid ingredient that is used to impart a sweet taste to food or drug product. Sweeteners are often classified as either nutritive (caloric) or non-nutritive (non-caloric), natural or synthetic. Examples of sweeteners include but are not limited to sucrose, dextrose, lactose, glucose, advantame, sorbitol, mannitol, liquid glucose, honey molasses, saccharin, sucralose, rebaudioside A stevia, rebaudioside M stevia, stevioside, mogroside IV, mogroside V, alitame, saccharin, neohesperidin dihydrochalcone, cyclamate, neotame, N-[3_(3-hydroxy-4-methoxybenzyl yl) propyl]-L-a-aspartyl]-L-phenylalanine 1-methyl ester, N-[3-(3-hydroxy-4-methoxyphenyl)-3-methylbutan yl]-L-a-aspartyl]-L-phenylalanine 1-methyl ester, N-[3-(3-methoxy-4-hydroxyphenyl) propyl]-L-a-aspartyl]-L-phenylalanine 1-methyl ester, curculin, cyclamate, aspartame, acesulfame potassium and others or mixtures thereof.

The term “plasticizer” refers to a component that reduces the glass-transition temperature of the film forming polymers (e.g., the water soluble polymer or water soluble polymers in the film). The plasticizer increases the flexibility, enhances elasticity and reduces brittleness of the film. Examples of plasticizers that can be used in the disclosed oral film dosage forms include triacetin, triethyl citrate, tributyl citrate, acetyl tributyl citrate, acetyl triethyl citrate, trioctyl citrate, acetyl trioctyl citrate, trihexyl citrate, dibutyl sebacate, PEG 300, PEG 400, Glycerine, etc. Plasticizer may be added in an amount up to 25%, alone or as a combination, of the total mass of the film oral dosage form, such as from 0.5% to 25%, 1% to 20%, 2% to 15% or 5% to 10%.

The term “preservative agent” refers to ingredient that is pharmaceutically acceptable to kill any bacteria and prevent mold growth that may result during drug storage examples comprising methyl propylparaben, propylparaben, benzalkonium chloride, propylene glycol, and benzoic acid.

The term “film forming polymer” refers to a polymer used in a film, affecting mechanical proprieties as elasticity, foldability and malleability. Examples of film forming polymers are copovidone (plasdone) and hypromelose HPMC E50.

Water soluble polymers that can be employed in the disclosed films include water soluble cellulose derivatives, including hydroxypropylmethyl cellulose, hydroxyethyl cellulose, hydroxypropyl cellulose; polyvinyl pyrrolidone (PVP); copovidone (a copolymer of 1-vinyl-2-pyrrolidone and vinyl acetate); other copolymers of vinyl pyrrolidone; other polymers or copolymers of substituted vinyl pyrrolidone; derivatives of polyvinyl pyrrolidone; polyethylene oxide, carboxymethyl cellulose; polyvinyl alcohol; natural gums, including xanthan, tragacanth, guar, acacia and arabic gums; and water soluble polyacrylates. Combinations of these water soluble polymers or other water soluble polymers can also be used. Examples of substituted vinyl pyrrolidones include N-vinyl-3-methyl-2-pyrrolidone, N-vinyl-4-methyl-2-pyrrolidone, N-vinyl-5-methyl-2-pyrrolidone, N-vinyl-5,5-dimethyl-2-pyrrolidone, N-vinyl-3,3,5-trimethyl-2-pyrrolidone and others. Examples of monomers that can be copolymerized with vinyl pyrrolidone or substituted vinyl pyrrolidones include vinyl aromatic monomers such as styrene, and acrylate or methacrylate monomers such as methyl methacrylate and 2-dimethylaminoethyl methacrylate.

As used herein, a film layer that is “unbuffered” is a film layer that does not contain a weak acid or weak base that is effective to maintain pH near a chosen value upon addition of another acid or base. Stated differently, the unbuffered film layer does not contain a buffering agent, such as borates, citrates, or phosphates.

The term “stabilized” as used herein refers to inhibition or retardation of changes of volume and/or loss of surface area, and/or increases in structural order of the plurality of active particles. More specifically, in the presence of certain macromolecules or polymers, the material shows an improved lifetime in an optimal particle size range, as characterized by reduced rate of agglomeration, increased structural order, crystallization and/or recrystallization of therapeutically active ingredient, as to demonstrate a desired solubilization profile in a preferred liquid medium.

The disclosed psilocybin dosage forms are formulated as orally administered films comprising psilocybin, or psilocybin analogs disposed within or on a polymeric film suitable for oral administration. The oral film formulations can be formulated for rapid disintegration and distribution of micro- or nanoscopic particles of the active agent in the gastrointestinal tract or as mucoadhesive films that facilitate rapid absorption of psilocybin via oral mucosal tissue, i.e., buccal or sublingual film dosage forms. The oral film formulations can also be formulated to have a longer residence time if desired based on various factors including the active agent being employed in a given formulation.

Preferred film dosage forms include sublingual and buccal film oral dosage forms. Buccal and/or sublingual mucosa absorption allows the drug to be absorbed directly into the blood stream skipping the hepatic metabolism. From a pharmaceutical formulation perspective this is particularly challenging, as the process of transmucosal permeation needs to be carefully optimized to obtain an acceptable pharmacokinetic profile. Tryptamines such as but not limited to psilocybin, and psilocin are more preferably administered bucally to reduce the incidence of first past metabolism when ingested enterally. The use of a long lasting oral film allowing the dissolving film to direct the active through the mucosa directly to the blood stream is desired to improve the absorption profile of the API and consequently improve bioavailability. The convenience of an oral film over tablets allows better patient compliance, as many individuals have difficulty swallowing or might not have water readily available.

The disclosure also encompasses pharmaceutical compositions comprising the at least one cannabinoid in combination with psilocybin and/or/psilocin, preferably formulated into pharmaceutical dosage forms, together with suitable pharmaceutically acceptable carriers, such as diluents, fillers, salts, buffers, stabilizers, solubilizers, etc. The dosage form may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended oral route of administration to a patient.

Most drugs are weak organic acids or bases, existing in un-ionized and ionized forms in an aqueous environment. The un-ionized form is usually lipid soluble and diffuses readily across cell membranes. The ionized (charged) form has low lipid solubility and thus cannot penetrate cell membranes easily.

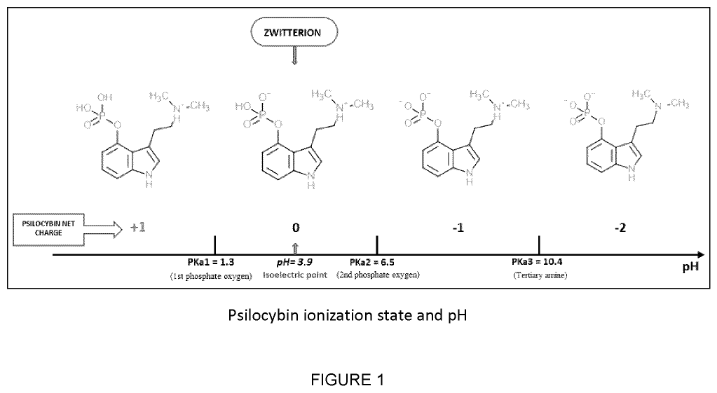

Psilocybin is a polar and hydrophilic drug. The pka values of the molecule are pka1=1.3 attributed to the first phosphate oxygen, pka2=6.5 attributed to the second phosphate oxygen and pka3=10.4 attributed to the tertiary amine group. Hence, the molecule carries negative and positive electrical charges and the total net charge increases and decreases depending on pH conditions. This explains the difficulty of the drug to penetrate oral mucosa membrane at the physiological pH of the mouth. However, there is an acidic pH range around the value 3.9 where the psilocybin molecule is neutral and does not carry a net charge as illustrated by FIG. 1.

It is disclosed that adjusting the pH of the buccal microenvironment that surrounds psilocybin film using an acidifying agent can modify the pH of the saliva in that area and reduce psilocybin ionization or psilocybin electrical charge, which will increase psilocybin permeability.

In fact, the use of phosphoric acid or citric acid in the film formulation enables psilocybin to be around its isoelectric point (pH=3.9) where the molecule is electrically neutral, which triggers permeation of psilocybin through pig mucosa membrane.

Saliva has a pH normal range of 6.2-7.6 with 6.7 being the average pH and has also a buffering capacity. As the contact time between psilocybin and saliva in the sublingual or buccal cavity increases, the pH of the microenvironment that surrounds psilocybin in the film moves slowly towards saliva pH value and the molecule starts to acquire negative electrical charge that will slow the permeation rate of psilocybin through the mucosa membrane.

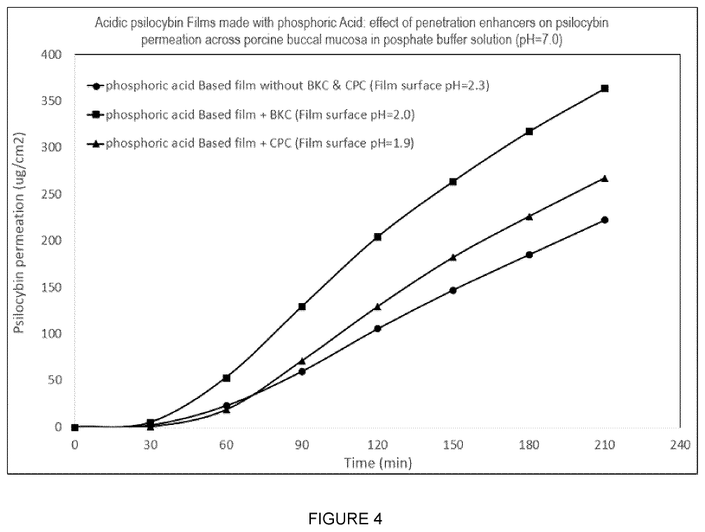

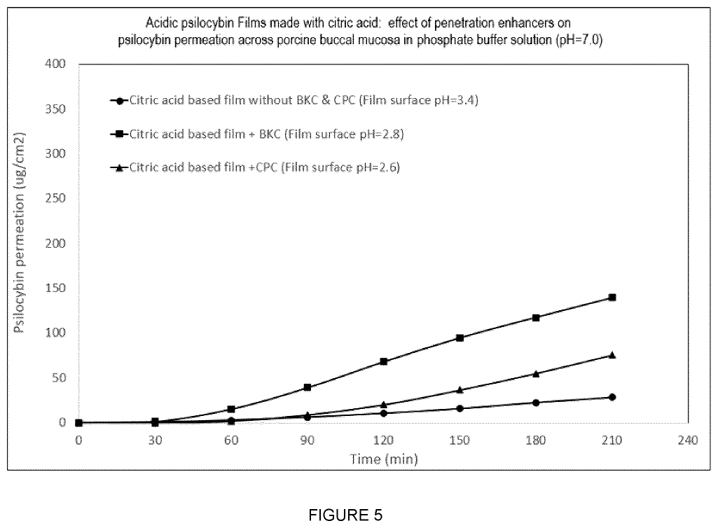

We hypothesized that adding in the formulation an appropriate lipophilic counter-ion such as cationic quaternary ammonium salts, can form an ion-pair in solution with the negatively charged psilocybin at the physiological pH of the mouth resulting in an electrically neutral and potentially more lipophilic entity that is able to permeate the muccosa membrane. In fact, the use of penetration enhancers such as Benzalkonium chloride (BKC) and cetylpyridinium chloride (CPC) in the film formulation improved buccal permeation of psilocybin as illustrated by FIG. 4 and FIG. 5.

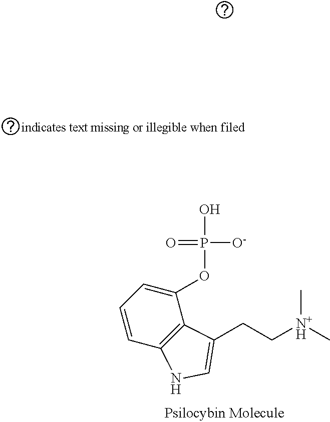

Table 1 shows derivatives of Psilocybin.

TABLE 1
Psilocybin Derivatives

| Derivative | Description |
|---|---|
| Psilocin | Dephosphorylated psilocybin. |
| Norpsilocin | Psilocin with one methyl in the ethyl amino group. |
| Aeruginascin | Psilocybin with three methyl groups in the ethyl |
| | amino group. |
| Baeocystin | Psilocybin with one methyl in the ethyl amino |
| | group. |
| Norbaeocystin | Psilocybin with no methyl groups on the ethyl |
| | amino group. |
| Bufotenin | Psilocin substituted on the 5^{th} carbon instead of the |
| | 4^{th}. |
| Bufotenidine | Bufotenin with three methyl groups on the ethyl |
| | amino group. |

One approach to address the needs described above is the use of penetration enhancer in the formulation to enhance psilocybin absorption. As disclosed herein, several penetration enhancers were evaluated at various concentrations. Only two molecules showed a significate increase in psilocybin diffusion through porcine buccal mucosa: Benzalkonium chloride (BKC) and cetylpyridinium chloride CPC) which belong to the cationic surfactants category.

The second approach to enhance psilocybin or psilocin absorption is the use of an acidifying agent such as phosphoric acid and citric acid that enable the psilocybin film to have a surface pH value between 1.5 and 5, which will be disclosed herein.

According to certain embodiments, the use of a quaternary ammonium cation (Benzalkonium chloride, Cetylpyridinium) and an acidifying agent (Phosphoric acid) in a psilocybin formulation has surprisingly increased the permeation of psilocybin through porcine buccal mucosa. Increasing the permeation of psilocybin in this case means that in a similar formulation without an acidifying agent that adjusts the pH within a desired range, or a penetration enhancer, the active agent would pass through the buccal mucosa to a lesser extent without the presence of said acidifying agent or penetration enhancer.

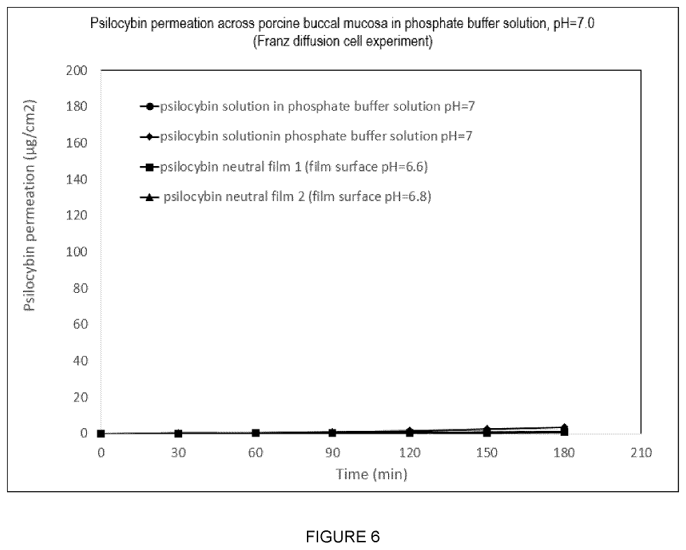

Before a buccal drug delivery system can be formulated, buccal absorption/permeation studies must be conducted to determine the feasibility of this route of administration. In the case of a psilocybin film, in vitro permeability studies were conducted in a 6 station Franz diffusion cell apparatus using buccal mucosa obtained from freshly slaughtered pigs. All the tests conducted in neutral media such as artificial saliva (pH=6.8), real saliva (pH=7.3) and phosphate buffer solution (pH=7) showed that psilocybin film did not diffuse through porcine buccal mucosa. In addition, psilocybin solutions prepared in the aforementioned media did not show any diffusion through porcine buccal mucosa as illustrated by FIG. 6.

According to certain embodiments, the disclosed oral films can comprise psilocybin, or psilocybin analogues disposed in or on a polymeric film-forming system and can beneficially include a flavoring agent, a sweetener, a permeation enhancer, an antioxidant, an acidifying agent or pH stabilizing system, or a combination of two or more thereof.

According to certain embodiments, pharmaceutically acceptable salts that may be used in the film dosage forms disclosed herein including generally any salt that has been or may be approved by the US FDA or other appropriate foreign or domestic agency for administration to a human. Non-limiting examples include hydrochloric, hydrobromic, nitric, carbonic, monohydrocarbonic, phosphoric, monohydrogen phosphoric, dihydrogen phosphoric acid, sulfuric acid, a hydrogen sulfuric acid, and hydroiodic acids of psilocybin. Other examples include salts derived from nontoxic organic acids, including acetic, propionic, isobutyric, maleic, malonic, benzoic, succinic, suberic, fumaric, lactic, mandelic, phthalic, benzenesulfonic, p-toluenesulfonic, citric, tartaric, and methanesulfonic acids, or combinations of these acid salts.

According to certain embodiments, the active psilocybin agent can comprise about 2% to 25% or 5% to 20% of the weight of the film on a dry basis.

According to certain embodiments, the polymeric film forming system can comprise a single pharmaceutically acceptable film-forming polymer or a combination of film-forming polymers. Examples of film-forming polymers that can be used for preparing the disclosed psilocybin dosage forms include copovidone (copolymers of N-vinyl-2-pyrrolidone and vinyl acetate), povidone (polyvinylpyrrolidone), polyethylene oxide, polyvinyl alcohol, polyethylene glycol, polyacrylic acid, methylmethacrylate copolymer, carboxyvinyl copolymers, polydextrose, pullulan, hydroxypropylmethyl cellulose, hydroxyethyl cellulose, hydroxypropyl cellulose, carboxymethyl cellulose, sodium alginate, xanthan gum, tragancath gum, guar gum, acacia gum, arabic gum, starch and gelatin.

The selection of film-forming polymers can be made to dissolve completely over a period of time that is sufficient to ensure delivery and absorption of a therapeutically effective amount of psilocybin via oral mucosa, yet not so long as to cause annoyance or discomfort to the subject being administered psilocybin. For example, a film dosage form can be formulated to reside in the buccal cavity or sublingual region for a period of from 1 minute to an hour, 10 minutes to 30 minutes, or 15 minutes to 30 minutes.

According to certain embodiments, the film-forming polymer or combination of film-forming polymers can comprise 10% to 90%, 20% to 80% or 30% to 70% of the weight of the film oral dosage form on a dry basis.

Because the taste of psilocybin is slightly bitter, it is beneficial to add a sweetener, flavoring agent, refreshing agent, taste-masking agent, or a combination of these materials. According to certain embodiments, examples of sweeteners that can be used in the disclosed psilocybin film dosage forms include acesulfame potassium, aspartame, aspartan-acesulfame salt, cyclamate, erythritol, glycerol, glycyrrhizin, hydrogenated starch hydrolysate, isomalt, lactitol, maltitol, mannitol, neotame, polydextrose, saccharin, sorbitol, sucralose, tagatose, xylitol, dextrose, glucose, fructose, and honey. Flavoring agents that can be added to the disclosed loxapine film dosage forms include isoamyl acetate (banana flavor), benzaldehyde (cherry flavor), cinnamaldehyde (cinnamon flavor), ethyl propionate (fruit flavor), methyl anthranilate (grape flavor), limonene (orange flavor), ethyl decadienoate (pear flavor), allyl hexanoate (pineapple flavor), ethyl meltol, ethylanillin (vanilla flavor), and methyl salicylate (wintergreen flavor). Refreshing agents, also called cooling agents, are chemicals that trigger the cold sensitive receptors creating a cold sensation. Refreshing agents that can be added to the loxapine film oral dosage forms disclosed herein include menthol, thymol, camphor and eucalyptol.

According to certain embodiments, sweeteners, flavoring agents, and refreshing agents can be added in conventional quantities, generally up to a total amount of 5% to 10% of the weight of the film on a dry basis, e.g., 0.1% to 10%, or 0.5% to 5%.

According to certain embodiments, the psilocybin film oral dosage forms disclosed herein can advantageously employ an antioxidant or oxygen scavenger to prevent or reduce oxidative degradation of the psilocybin, or psilocybin analogs prior to use. Examples of oxygen scavengers or antioxidants that substantially improve long-term stability of a psilocybin film oral dosage form against oxidative degradation of the active agent are propyl gallate, EDTA, Copper II chloride, Vitamin E and Sodium metabisulfite,

According to certain embodiments, a suitable amount of antioxidant or chelating agent is from about 0.01% to 5% or 0.1% to 1% of the weight of the film on a dry basis.

It was discovered that absorption of psilocybin through oral mucosa occurred when the surface pH of the film is maintained at a surface acidic surface pH of from 1.5 to 5. Because the blend of psilocybin, film forming polymers and other ingredients tend to create a neutral pH, it is beneficial to add an acidifying substance that decreases the pH of the film product and stabilizes the film at an acidic pH. Examples of acidifying agents that can be added to the films disclosed herein include phosphoric acid, citric acid, tartaric acid, malic acid, acetic acid and succinic acid, hydrochloric acid, maleic acid, glucuronic acid, lactic acid preferably phosphoric acid or citric acid.

According to certain embodiments, phosphoric acid or other acidifying agents can be added to the psilocybin film oral dosage forms disclosed herein in amounts effective to adjust the pH within a range of from 1.5 to 5.0, with a suitable amount being, for example, 0.5% to 15% or 1% to 10% based on the weight of the film on a dry basis.

The surface pH value of a dry film can be measured by applying 1 or 2 drops of DI water and placing a flat glass pH electrode on the moistened surface. Allow sufficient time for stabilization of pH meter (30 to 60 seconds) and record pH value.

To further promote absorption of psilocybin through oral mucosa and reduce the amount of psilocybin that is introduced into the gastrointestinal tract, it is advantageous to add to the psilocybin film formulation a penetration enhancer. It has been discovered that particularly effective penetration enhancers that promote absorption of psilocybin via oral mucosa are benzalkonium chloride, cetylpyridinium chloride and other quaternary ammonium salts. Preferred penetration enhancers are selected from but not limited to cationic surfactants groups including benzalkonium chloride (BKC), cetylpyridinium chloride (CPC), Cetrimide and Cetyltrimethylammonium bromide (CTAB).

A penetration enhancer, such as benzalkonium chloride or cetylpyridinium chloride, can be added to the psilocybin film oral dosage form in an amount of from about 0.1% to about 10%, 0.5% to 5%, or 1% to 3% of the weight of the film dosage form on a dry basis to significantly enhance absorption of psilocybin from the film through oral mucosa.

In order to promote adhesion of the psilocybin film oral dosage form to oral mucosa, it is advantageous to add a mucoadhesive agent to the film product. Examples of mucoadhesive agents that can be added to the psilocybin film oral dosage form to promote adhesion to oral mucosa include hydroxypropylmethyl cellulose, hydroxyethyl cellulose, hydroxypropyl cellulose, sodium alginate, sodium carboxymethyl cellulose, guar gum, karya gum, methylcellulose, polyethylene oxide, retene and tragacanth. According to certain embodiments, such mucoadhesive agents may be added to the film formulation in an amount of from about 0.5% to about 20%, or about 1% to about 5%, of the total weight of the film on a dry basis.

Plasticizers can be advantageously employed in the film formulations as needed to suitably modify the flexibility of the film to facilitate processing and allow the film to easily conform to the shape of the oral mucosa to which the film is applied. Plasticizers that can be effectively employed in the disclosed psilocybin film oral dosage forms to improve flexibility of the film can be selected from ethylene glycol, propylene glycol, tributyl citrate, triethyl citrate, glycerol, and combinations of two or more thereof. Depending on the selected film-forming polymers and other components of the film formulation, a suitable amount of plasticizer is typically from about 0.1% to 10%, 0.5% to 5%, or 1% to 5%.

Bulking agents or fillers may be added as desired to increase the size of the finished film product to facilitate processing and manufacturing, or to modify properties (e.g., increase or decrease residence time or increase stiffness) of the film formulation. Suitable fillers that can be added to the disclosed film products include starch, calcium salts, such as calcium carbonate, and sugars, such as lactose. The amount of fillers that can be added to the film oral dosage forms disclosed herein are typically up to about 25%, 0.5% to 20%, 1% to 15% or about 2% to about 10% of the weight of the film on a dry basis.

In Table 2, it is disclosed the first example of a psilocybin film formulation using phosphoric acid as the acidifying agent and benzalkonium chloride as the penetration enhancer.

TABLE 2
Composition of psilocybin film made with phosphoric
acid as acidifying agent and benzalkonium
chloride (BKC) as penetration enhancer.

| | % wet | % dry | |
|---|---|---|---|
| Ingredients (g) | (w/w) | (w/w) | Function |
| water | 75.95 | — | Solvent |
| Phosphoric acid | 1.11 | 5.07 | Acidifying agent |
| Psilocybin | 1.52 | 6.89 | Active |
| Benzalkonium chloride | 0.51 | 2.34 | Penetration enhancer |
| Sucralose | 0.10 | 0.47 | Sweetener |
| D-Maltitol | 1.54 | 7.01 | Disintegrating agent |
| | | | (and pore former) |
| L-Menthol | 0.26 | 1.17 | Flavoring & taste |
| | | | masking agent |
| Glycerin | 2.06 | 9.35 | Plasticizer |
| Copovidone (e.g., | 10.27 | 46.68 | Film forming polymer |
| plasdone -630) | | | |
| hydroxypropyl | 3.70 | 16.82 | Film forming polymer |
| methylcellulose | | | |
| (e.g., HPMC E50) | | | |
| hydroxypropyl | 0.92 | 4.21 | Mucoadhesive agent |
| methylcellulose - | | | (and film former) |
| (e.g., HPMC E5) | | | |
| Methanol | 2.06 | — | solvent |
| Total | 100 | 100 | |

In Table 3, it is disclosed the second example of a psilocybin film formulation using citric acid as the acidifying agent and cetylpyridinium chloride as the penetration enhancer.

TABLE 3
Composition of psilocybin film made with citric acid as acidifying
agent and cetylpyridinium chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Psilocybin | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., | 9.80 | 41.76 | Film forming polymer |
| plasdone-630) | | | |
| hydroxypropyl | 3.66 | 15.58 | Film forming polymer |
| methylcellulose | | | |
| (e.g., HPMC E50) | | | |
| hydroxypropyl | 1.32 | 5.63 | Mucoadhesive agent |
| methylcellulose | | | |
| (e.g., HPMC E5) | | | |
| Methanol | 2.54 | — | solvent |
| Total | 100 | 100 | |

Referring to table 4, the selection of excipients are disclosed and their functions explained.

TABLE 4
Selection of Excipients in Psilocybin Film Formulation

| Component/Grade | Function | Justification |
|---|---|---|
| Glycerin USP | Plasticizer | Psilocybin film is a polymeric film that needs some |
| | | plasticizer to have good mechanical properties. |
| | | Glycerin is added to the formulation to improve the |
| | | softness and reduce the brittleness of the film. |
| Sucralose USP | Sweetener | Sucralose has a great sugar-like taste that can help |
| | | masking psilocybin bitter taste in the film. |
| D-Maltitol NF | Pore former, | D-maltitol is freely soluble in water. Hence, it is used as |
| | disintegrating | a pore former in the film helping the matrix of the film to |
| | agent | hydrate and disintegrate upon contact with saliva. |
| Psilocybin | Drug substance | Psilocybin is the active pharmaceutical ingredient |
| | | aimed to treat mental health disorders such anxiety and |
| | | depression. |
| L-menthol USP | Flavoring agent | The drug substance psilocybin has a bitter taste. L- |
| | | menthol helps to mask the bitterness in the film and |
| | | improve patient compliance |
| Copovidone (e.g., | Film forming | Copovidone is the main film forming polymer in |
| Plasdone S630) | polymer | psilocybin film. The amount used enables the film to |
| USP, EP, JPE | | have good mechanical proprieties as elasticity, good |
| | | folding characteristics and malleability |
| hydroxypropyl | Film forming | hydroxypropyl methylcellulose mid viscosity is used as |
| methylcellulose | polymer | a secondary film forming polymer to increase blend |
| (e.g., HPMC mid- | | viscosity since the two other film forming polymers have |
| viscosity) USP, Ph. | | low viscosity in solution. |
| Eur., JP | | |
| | | hydroxypropyl methylcellulose low viscosity is used as |
| | | a tertiary film forming polymer. It helps to have a fast |
| hydroxypropyl | Film forming | hydration of the film in the mouth which improves film |
| methylcellulose | polymer and | mucoadhesion and film retention time in the mouth. |
| (e.g., low viscosity | mucoadhesive | |
| HPMC) USP. EP, | agent | |
| JP and ChP | | |
| Phosphoric acid | acidifying agent, | Phosphoric acid play critical roles in the film formulation |
| (H₃PO₄) NF | solubilizer and | 1-Acidifier: Psilocybin film is designed for buccal |
| | | absorption in the mouth. Permeability studies with fresh |
| | | pig mucosa showed that diffusion of the drug through |
| | | porcine mucosa only happens with acidic films. The |
| | | proportion of phosphoric acid used in the film formula |
| | | is necessary to produce a film with a surface pH = 2 that |
| | | is optimal for buccal absorption |
| | | 2-Solubilizer: phosphoric acid solubilises and maintains |
| | | the psilocybin in solution during blend preparation |
| | | 3-Stabilizer: |
| | Stabilizer | Stability studies showed that psilocybin films made with |
| | | phosphoric acid do not need the presence of anti- |
| | | oxidant agents and produced the best impurity profile |
| | | after storing the films at 50° C. for two weeks |
| Purified water USP | Solvent | — |
| Methanol NF | Solvent | Since L-menthol is insoluble in water, small amount of |
| | | methanol is used to dissolve L-menthol in order to |
| | | disperse it into the blend during blend preparation. |
| | | Methanol is then evaporated during the drying process. |

The foregoing examples of film formulation are illustrative of the present invention. The resulting films showed considerable increase of psilocybin permeation across pig buccal mucosa as is also illustrated in FIGS. 4 and 5.

According to some embodiments, the tryptamine oral film dosage form has a residence time of between 5 and 60 minutes, preferably between 10 and 50 minutes and more preferably between 15 and 45 minutes. Therefore administering tryptamines as a film dosage form having a matrix for transmucosal delivery will remain in a patient mouth for a period of between 5 and 60 minutes, preferably between 10 and 50 minutes and more preferably between 20 and 40 minutes.

According to some embodiments, the exemplary psilocybin oral dosage forms disclosed herein can be prepared by dissolving or finely dispersing psilocybin and film forming polymers in a solvent, along with any other desired additives. Such additives include, but are not limited to, an acidifying agent, an antioxidant, a plasticizer, a penetration enhancer, a mucoadhesive agent, a flavoring agent, a coloring agent, a freshening agent, a sweetener, a filler, or a combination of additives. The films may then be cast on a suitable substrate by removing (e.g., evaporating) the solvent or solvents from the formulation to produce a dry film. Typically, the psilocybin film can be cast to produce a film having a thickness of from 100 micrometers to 1500 micrometers or 500 micrometers to 1000 micrometers. The dry film can be cut in appropriate sizes, typically an area of from about 1 square centimeter to about 15 square centimeters, to provide an appropriate dose for transmucosal delivery of psilocybin, to treat depression, anxiety, or addictive disorders.

According to some other embodiments, the tryptamine oral film comprises tryptamine active or derivatives suspended in amorphous form within the oral film matrix.

According to some other embodiments, more than one tryptamine is present in the oral film formulation.

Although the present disclosure outlined the preferred approach of having a solubilized tryptamine active or tryptamine derivative, such as psilocybin or psilocin, within the oral film matrix to promote buccal absorption, an amorphous tryptamine dispersed in a suspended particulate form within the oral film dosage form is also disclosed.

Numerous formulation strategies have been investigated to overcome the poor absorption of psilocybin and related drug substances and achieving an effective load of this API in the oral film. Throughout our experimentation permeation could not be achieved without the presence of an acidic environment. In addition the presence of a permeation enhancer was also critical to obtain buccal permeation. Both Psilocybin film and psilocybin solution showed a diffusion through porcine buccal mucosa at low pH.

Hence, it is important to develop a low pH film of psilocybin and test its diffusion through porcine buccal mucosa in a neutral medium (saliva). In neutral pH solution, psilocybin is not able to permeate through porcine buccal mucosa.

Successful approaches are achieved for formulation when the API is dispersed in film matrix, or when the API is solubilized. Other aspects of the formulation such as film matrix physical properties, disintegration rate, drug substance effective surface area and solubility were also considered to improve the film dissolution rate and thus the API bioavailability.

The oral film formulations are used to orally administer a tryptamine active. This is accomplished by preparing the films as described above and introducing them to the oral cavity of a human or animal, such as a mammal. This oral film may be prepared and adhered to a second or support layer from which it is removed prior to use, i.e. introduction to the oral cavity. An adhesive may be used to attach the oral films to the support or backing material which may be any of those known in the art, and is preferably not water soluble. If an adhesive is used, it will desirably be a pharma-grade material that is ingestible and does not alter the properties of the active.

When designed for animal administration, the oral films may desirably be designed to adhere to the oral cavity of the animal including the tongue, thus preventing it from being ejected from the oral cavity and permitting more of the active to be introduced to the oral cavity as the film disintegrates.

Another use for the oral films of the present disclosure takes advantage of the tendency of the film to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

According to embodiments, the tryptamine oral film was formed as a monolayer and is applied within the oral cavity where it adheres to the oral mucosa, and facilitates API to permeation through the oral membrane and entering the underlying blood capillaries. As the film is designed as a monolayer, when it is applied to the mucosa surface one face of the film is exposed to saliva and abrasive rubbing. This may lead to premature solubilization of the film and swallowing of the API, which may decrease the efficacy and consistency of the film in reaching the target pharmacokinetic profile. Accordingly the present further embodiments are designed for using a multilayer strategy to protect the film from premature solubilization and favor mucosal directed oral permeation and uptake of the API.

The disclosed film can be used in a monolayer form, or in a multilayer form. In particular, a barrier layer can be advantageously employed to prevent the active agent from diffusing through a bioadhesive film into the oral cavity of a subject after it is adhered to the subject's oral mucosa. The barrier layer is preferably comprised of polymers having a low solubility in water. A combination of water-insoluble polymer(s) and a minor amount of a water-soluble polymer(s) can be employed to maintain a barrier that prevents loss of the active agent to the oral cavity until an effective or desired amount of the active agent has been transmucosally delivered, and which allows erosion and/or dissolution thereafter. In some cases it may be advantageous to employ, in the barrier layer, higher molecular weight polymer analogs of the polymer(s) used in the bioadhesive layer. The higher molecular weight (or, equivalently, higher viscosity) analogs are typically more resistant to diffusion and dissolution, and exhibit better compatibility than if polymers of a different chemical type are used.

Examples of water-insoluble polymers that can be employed in the barrier layer include polysiloxanes (silicone polymers), ethyl cellulose, propyl cellulose, polyethylene, and polypropylene. One or more of these polymers may comprise a majority of the barrier film layer by weight (i.e., at least 50 percent). Water soluble hydroxypropyl cellulose can be used in a minor amount to facilitate erosion and/or dissolution of the barrier layer after it has served its function during transmucosal delivery of the active agent. High viscosity polymer could also be used to create a barrier and limit erosion. For example, hydroxypropyl cellulose, polyethylene oxide, polyvinyl pyrrolidone and any other polymer soluble in water, but exhibiting high viscosity, can be used.

Uptake of API in the oral cavity using film based pharmaceuticals can be surprisingly improved using a multilayer film strategy. In its simplest embodiment, a multilayer film consists of at least two layers. The first layer which contains the API and mucoadhesive polymers to ensure adhesion and close contact with the oral mucosa. The second layer, or backing layer, serves to slow the solubilization of the film by limiting the wetting of the active layer, while at the same time reducing abrasion from the tongue and cheek. In this way, we direct and favor absorption of the API from the active layer into the oral mucosa and underlying capillaries, rather than having the active layer quickly disintegrated and swallowed. This multilayer strategy is not limited to a bilayer system, and could potentially incorporate other multilayers that may serve to further protect the active layer in modular time scales or for other purposes. Furthermore, the protective layer could be composed of variable % w/w of high molecular polymers to control the rate of disintegration and residence time in the oral cavity. Examples of high MW polymers that would be effective include but are not limited to HPC, HPMC, HEC, PVP, PVA or PEO.

The protective backing layer can be used to further increase the local concentration at the membrane interface and minimize ionization of the active agent due to the local pH environment within the oral cavity.

The pH within the mouth typically ranges from pH 6-7.5. Therefore, as a monolayer film is placed in the mouth and begins to solubilize, tryptamines that are not absorbed directly into the oral mucosa will be exposed to the lower pH environment, precipitate and be swallowed. Maintaining amorphous solubilized tryptamines is critical for ensuring the target pharmacokinetics. Therefore, in one embodiment a multilayer system could further improve the tryptamine active oral film performance by maintaining the local pH environment around the active film, which would improve tryptamines such as psilocybin absorption in the oral cavity. The multilayer in this embodiment could also be designed with acidic buffering agents or general acidifying excipients such as phosphoric acid.

In another embodiment of the multilayer platform, the oral consist of an active layer directly placed against the mucosa, a middle layer serving to protect the active layer from oral environment, and a third layer containing acidifying agents or acid buffering components to maintain the pH below 5. This strategy of maintaining the pH environment can also be extended to basic pH environments which could improve both solubility and stability for tryptamines that could have different pKa profiles than psilocybin and its derivatives.

An exemplary multilayer tryptamine oral film formulation is illustrated in Table 5:

TABLE 5
Exemplary Multilayer Tryptamine Oral
Film Formulation with psilocybin

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Psilocybin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 6
Exemplary Multilayer Tryptamine Oral
Film Formulation with Psilocin

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Psilocin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 7
Exemplary Multilayer Tryptamine Oral
Film Formulation with Norpsilocin

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Norpsilocin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 8
Exemplary Multilayer Tryptamine Oral
Film Formulation with Bufotenin

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Bufotenin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 9
Exemplary Multilayer Tryptamine Oral
Film Formulation with Bufotenidine

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Bufotenidine | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 10
Exemplary Multilayer Tryptamine Oral
Film Formulation with Baeocystin

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Baeocystin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 11
Exemplary Multilayer Tryptamine Oral Film Formulation with

| | First layer | Second layer |
|---|---|---|
| | (Mucoadhesive layer) | (Backing layer) |
| Ingredients | % dry | % dry |
| water | — | — |
| Phosphoric acid | 5.07 | — |
| Norbaeocystin | 6.89 | — |
| Benzalkonium chloride | 2.34 | — |
| Sucralose | 0.47 | 0.40 |
| D-Maltitol | 7.01 | 5.96 |
| L-Menthol | 1.17 | 0.99 |
| Glycerin | 9.35 | 7.95 |
| Copovidone | 46.68 | 39.70 |
| (e.g., plasdone S-630) | | |
| Hydroxypropyl | 16.82 | 45.00 |
| methylcellulose | | |
| (e.g., HPMC E50) | | |
| Hydroxypropyl | 4.21 | — |
| methylcellulose | | |
| (e.g., HPMC E5) | | |
| Methanol | — | — |
| Total | 100 | 100 |

TABLE 12
Composition of Psilocin film made with citric acid as acidifying
agent and cetylpyridinium chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Psilocin | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., plasdone- | 9.80 | 41.76 | Film forming polymer |
| 630) | | | |
| hydroxypropyl | 3.66 | 15.58 | Film forming polymer |
| methylcellulose (e.g., | | | |
| HPMC E50) | | | |
| hydroxypropyl | 1.32 | 5.63 | Mucoadhesive agent |
| methylcellulose (e.g., | | | |
| HPMC E5) | | | |
| Methanol | 2.54 | — | solvent |
| Total | 100 | 100 | |

TABLE 13
Composition of Bufotenin film made with citric acid as acidifying
agent and cetylpyridinium chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Bufotenin | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., plasdone- | 9.80 | 41.76 | Film forming polymer |
| 630) | | | |
| hydroxypropyl | 3.66 | 15.58 | Film forming polymer |
| methylcellulose (e.g., | | | |
| HPMC E50) | | | |
| hydroxypropyl | 1.32 | 5.63 | Mucoadhesive agent |
| methylcellulose (e.g., | | | |
| HPMC E5) | | | |
| Methanol | 2.54 | — | solvent |
| Total | 100 | 100 | |

TABLE 14
Composition of Bufotenidine film made with citric acid as acidifying
agent and cetylpyridinium chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Bufotenidine | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., plasdone- | 9.80 | 41.76 | Film forming polymer |
| 630) | | | |
| hydroxypropyl | 3.66 | 15.58 | Film forming polymer |
| methylcellulose (e.g., | | | |
| HPMC E50) | | | |
| hydroxypropyl | 1.32 | 5.63 | Mucoadhesive agent |
| methylcellulose (e.g., | | | |
| HPMC E5) | | | |
| Methanol | 2.54 | — | solvent |
| Total | 100 | 100 | |

TABLE 15
Composition of Norpsilocin film made with citric acid as acidifying
agent and cetylpyridinium chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Norpsilocin | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., plasdone- | 9.80 | 41.76 | Film forming polymer |
| 630) | | | |
| hydroxypropyl | 3.66 | 15.58 | Film forming polymer |
| methylcellulose (e.g., | | | |
| HPMC E50) | | | |
| hydroxypropyl | 1.32 | 5.63 | Mucoadhesive agent |
| methylcellulose (e.g., | | | |
| HPMC E5) | | | |
| Methanol | 2.54 | — | solvent |
| Total | 100 | 100 | |

TABLE 16
Composition of Norbaeocystin film made with citric
acid as acidifying agent and cetylpyridinium
chloride (CPC) as penetration enhancer.

| | % wet | % dry | |
|---|---|---|---|
| Ingredients (g) | (w/w) | (w/w) | Function |
| water | 73.99 | — | Solvent |
| Citric acid | 2.19 | 9.35 | acidifying agent |
| Norbaeocystin | 0.76 | 3.25 | Active |
| Cetypyridinium chloride | 0.76 | 3.25 | Penetration enhancer |
| Sucralose | 0.15 | 0.65 | Sweetener |
| D-Maltitol | 1.02 | 4.33 | Disintegrating agent |
| L-Menthol | 1.78 | 7.57 | Flavoring & taste |
| | | | masking agent |
| Polyethylene glycol | 2.03 | 8.65 | Plasticizer |
| (PEG300) | | | |
| Copovidone (e.g., plasdone- | 9.80 | 41.76 | Film forming polymer |
| 630) | | | |
| hydroxypropyl | 3.66 | 15.58 | Film forming polymer |
| methylcellulose (e.g., | | | |
| HPMC E50) | | | |
| hydroxypropyl | 1.32 | 5.63 | Mucoadhesive agent |
| methylcellulose (e.g., | | | |
| HPMC E5) | | | |
| Methanol | 2.54 | — | solvent |
| Total | 100 | 100 | |

According to some aspect of the present disclosure, the longer the residence time of the oral film within the oral cavity is achieved by increasing the viscosity of the polymers contained in the oral film matrix.

As disclosed herein, the dissolution time is the time taken to release the drug in solution whereas the dinstegration time is the time the oral film dosage form takes to break apart in pieces following the administration of the film in the buccal cavity.

Also disclosed herein is the use of the tryptamine oral film for the treatment of mental health disease such as addiction, depression, anxiety and posttraumatic stress disorder (PTSD).

Another use for the oral films of the present disclosure takes advantage of the tendency of the film to jellify quickly when in contact with liquids like saliva.

According to some embodiments, it is disclosed a tryptamine containing oral film dosage form or an oral film comprising a tryptamine active or derivative thereof wherein the oral film is preferably made from an aqueous blend.

According to some embodiments, it is disclosed a tryptamine containing oral film dosage form or an oral film comprising a tryptamine active or derivative thereof wherein the oral film is made from or comprise at least one film layer made from an organic solvent blend. The organic solvent or solvent based oral film formulation is desired to promote dissolution of the active within the blend prior to coating and drying of the oral film.

The oral films of the present disclosure are desirably packaged in sealed, air and moisture resistant packages to protect the active from exposure oxidation, hydrolysis, volatilization and interaction with the environment. Moreover, the films of the present invention dissolve quickly upon contact with saliva or mucosal membrane areas, eliminating the need to wash the dose down with water.

Desirably, a series of such unit doses are packaged together in accordance with the prescribed regimen or treatment, e.g., a 3-90 day supply, depending on the particular therapy. The individual films can be packaged on a backing and peeled off for use.

Manufacturing Procedure:

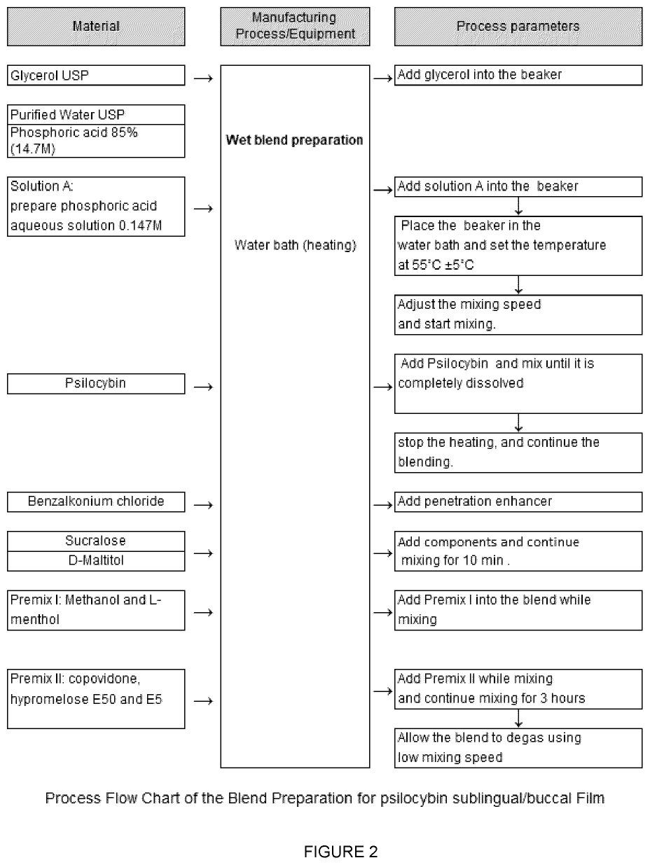

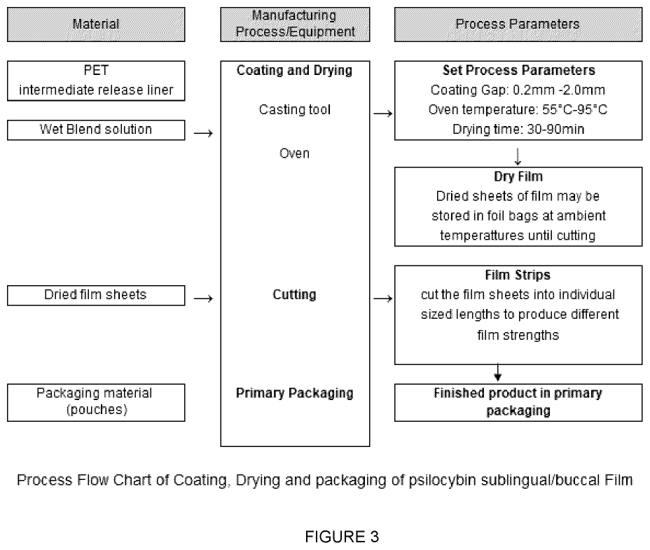

As illustrated in FIGS. 2 and 3, the currently disclosed oral film is produced by dissolving all the soluble ingredients in the media of choice and mixing until all excipients are solubilized yielding a uniform blend. The active ingredient is then added to the blend and solubilized or dispersed, depending on the formulation. The blend is mixed until uniform and then polymers are added to the blend and mixed until completely dissolved. Once dissolved and homogenous, the mixing speed is lowered to allow degassing of the blend.

Preparation of an oral film product typically involves casting or otherwise thinly spreading the liquid film formulation on a substrate, drying (e.g., evaporating) all or most of the solvent(s) from the film to produce a thin, semi-solid/solid film sheet of material, and cutting the film sheet into individual unit dosage forms for packaging or processing.

The oral film formulation of the present disclosure may be formed into a sheet prior to drying. After the desired components are combined to form a multi-component matrix, including the polymer, water, active other components as desired, the combination is formed into a sheet or film, by any method known in the art such as, coating, spreading, casting or drawing the multi-component matrix. A multi-layered film may be achieved by coating, spreading, or casting a combination onto an already formed film layer. Although a variety of different film-forming techniques may be used, it is desirable to select a method that will provide a flexible OF, such as reverse roll coating. The flexibility of the oral film allows for the sheets of oral film to be rolled and transported for storage or prior to being cut into individual dosage forms. Desirably, the oral film will also be self-supporting or in other words able to maintain their integrity and structure in the absence of a separate support. Furthermore, the films of the present invention may use selected materials that are edible or ingestible.

Coating or casting methods are particularly useful for the purpose of forming OF as disclosed herein. Specific examples include reverse roll coating, forward roll coating, gap or knife over roll coating, air knife coating, curtain coating, or combinations thereof, especially when a multi-layered film is desired.

Roll coating, or more specifically reverse roll coating, is particularly desired when forming films in accordance with the present disclosure. This procedure provides excellent control and uniformity of the resulting films, which is desired in the present disclosure. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The above description is considered that of the preferred embodiment(s) only. Modifications of these embodiments will occur by those skilled in the art and by those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film formulation comprising an active agent selected from psilocybin, psilocin, norpsilocin, bufotenin, bufotenidine, baeocystin, norbaeocystin, and aeruginascin, and salts thereof, and combinations thereof, a penetration enhancer, an acidifying agent, and a plasticizer, wherein the surface pH range of the oral film formulation is preferably between 1.5 and 5.

2. The oral film formulation of claim 1, wherein the surface pH range is between 2 and 4.5.

3. The oral formulation of claim 1, wherein the surface pH range is between 2.5 and 4.

4. The oral formulation of claim 1, wherein the surface pH range is between 3 and 3.5.

5. The oral film formulation of claim 3, wherein the penetration enhancer is a quaternary ammonium salt selected from benzalkonium chloride, cetylpyridinium chloride and Cetrimide, Cetyltrimethylammonium bromide.

6. The oral film formulation of claim 1, wherein the acidifying agent is selected from phosphoric acid, citric acid, tartaric acid, malic acid, acetic acid, succinic acid, maleic acid, Hydrochloric acid, benzoic acid, fumaric acid, glucoronic acid, or lactic acid, and is preferably citric acid or phosphoric acid.

7. The oral film formulation of claim 1, wherein the penetration enhancer is present in an amount of from about 0.1% to about 10% by weight, 1% to 3%, or preferable 0.5% to 5% by weight relative to the total dry weight of the oral film formulation.

8. The oral film formulation of claim 1, wherein the active agent is present in an amount of from about 2 to 25% by weight or 5 to 20% by weight, relative to the total dry weight of the oral film formulation.

9. The oral film formulation of claim 1, further comprising a mucoadhesive agent, wherein the mucoadhesive agent is selected from the group of hydroxypropylmethyl cellulose, hydroxyethyl cellulose, hydroxypropyl cellulose, sodium alginate, sodium carboxymethyl cellulose, guar gum, karya gum, methylcellulose, polyethylene oxide, retene, tragacanth, and combinations of two or more thereof.

10. The oral film formulation of claim 9, wherein the mucoadhesive agent is present in an amount of from about 0.5% to about 20% by weight, or about 1% to about 5% by weight, relative to the total dry weight of the oral film formulation,

11. The oral film formulation of claim 1, wherein the plasticizer is present in an amount up to 25% by weight, such as from 0.5% to 25%, 1% to 20%, 2% to 15% or 5% to 10% by weight, relative to the total dry weight of the oral film formulation.

12. The oral film formulation of claim 1, further comprising an antioxidant, wherein the antioxidant is present in an amount of from about 0.01% to 5% or 0.1% to 1% by weight, relative to the total dry weight of the oral film formulation.

13. The oral film formulation of claim 1, further comprising at least one film forming polymer, wherein the at least one film forming polymer is selected from the group of hypromelose HPMC E50, copovidone (copolymers of N-vinyl-2-pyrrolidone and vinyl acetate), povidone (polyvinylpyrrolidone), polyethylene oxide, polyvinyl alcohol, polyethylene glycol, polyacrylic acid, methylmethacrylate copolymer, carboxyvinyl copolymers, polydextrose, pullulan, hydroxypropylmethyl cellulose, hydroxyethyl cellulose, hydroxypropyl cellulose, carboxymethyl cellulose, sodium alginate, xanthan gum, tragancath gum, guar gum, acacia gum, arabic gum, starch, gelatin, and combinations of two or more thereof.

14. The oral film formulation of claim 13, wherein the film forming polymer or combination of film forming polymers is present in an amount of from 10% to 90% by weight, 20% to 80% by weight, or 30% to 70% by weight, relative to the total dry weight of the oral film formulations.

15. (canceled)

16. (canceled)

17. The oral film formulation of claim 1, further comprising a pore former.

18. (canceled)

19. The oral film formulation of claim 1, further comprising a sweetener.

20. A method for treating depression, anxiety, migraines, addiction, dementias, Alzheimer's disease, eating disorders, obsessive compulsive disorder, Lyme disease syndrome, post-traumatic stress disorder, abuse disorders including opioid addiction, alcohol addiction, nicotine addiction, cannabinoid addiction, headache, central nervous system inflammation, and disorders of cognition and memory with the oral film formulation of claim 1, wherein the method comprises administrating the oral film formulation bucally.

21. A method of using an acidifying agent and penetration agent for enhancing permeation of psilocybin, psilocin, norpsilocin, bufotenin, bufotenidine, baeocystin, norbaeocystin, and aeruginascin, and salts thereof, and combinations thereof, wherein the method comprises administering the oral film formulation buccally.

22. An oral film formulation comprising an active agent, a penetration enhancer, an acidifying agent and a plasticizer, wherein the pH range of the oral film formulation is preferably between 1.5 and 5, and wherein the active agent is a tryptamine.

23. A method for treating depression, anxiety, migraines, addiction, dementias, Alzheimer's disease, eating disorders, obsessive compulsive disorder, Lyme disease syndrome, post-traumatic stress disorder, abuse disorders including opioid addiction, alcohol addiction, nicotine addiction, cannabinoid addiction, headache, central nervous system inflammation, and disorders of cognition and memory with the oral film formulation of claim 22, wherein the method comprises administrating the oral film formulation buccally.

24. (canceled)
